(19) United States
(12) Patent Application Publication
(10) Pub. No.: US 2026/0060610 A1
(43) Pub. Date: Mar. 5, 2026

(54) SLEEP MANAGEMENT RING

(71) Applicant: HAPPY HEALTH, INC., Austin, TX (US)

(72) Inventors: Dustin M. FRECKLETON, Austin, TX (US); Byron P. OLSON, Boone, IA (US); Nithin O. RAJAN, Austin, TX (US); David E. CLIFT-REAVES, Austin, TX (US)

(21) Appl. No.: 19/383,484

(22) Filed: Nov. 7, 2025

Related U.S. Application Data
(63) Continuation of application No. 17/619,653, now abandoned.
(60) Provisional application No. 62/862,427, filed on Jun. 17, 2019.

(51) Int. Cl. A61B 5/00; A61B 5/0205; A61B 5/08; A61M 21/02; G16H 20/70

(52) U.S. Cl. CPC A61B 5/4815; A61B 5/02055; A61B 5/0816; A61B 5/6802; A61M 21/02; G16H 20/70

(57) ABSTRACT

Sleep management ring includes one or more physiological sensors configured to detect one or more physiological indicators of a user. An output device includes one or more context sensors. One or more processors executing instructions, on a memory to: receive one or more indicators of a bedtime from the context sensors; receive the physiological indicators; recommend a sleep preparation exercise for a user to perform; determine a change in the one or more physiological parameters and/or the one or more indicators of the bedtime after recording the sleep preparation exercise; and adjust the sleep preparation exercise based on one of the effectiveness of the sleep preparation exercise and the one or more physiological indicators as measured by the one or more physiological sensors of the wearable device.

### CROSS-REFERENCE TO RELATED APPLICATIONS

This patent application is a continuation of and claims priority to U.S. application Ser. No. 17/619,653, filed Dec. 16, 2021, entitled WEARABLE DEVICE OPERABLE TO DETECT AND/OR PREPARE A USER FOR SLEEP, which is a National Stage Entry of PCT/US2020/038237, filed Jun. 17, 2020, which claims the benefit of U.S. Provisional Application No. 62/862,427, filed on Jun. 17, 2019, the contents of each are incorporated by reference herein in their entirety.

### FIELD

The present inventive concept relates generally a wearable device operable to detect physiological measurements.

### BACKGROUND

Wearable devices are prominent in society and provide users with multiple data points regarding their physiological status.

### BRIEF DESCRIPTION OF THE DRAWINGS

The novel features of the disclosure are set forth with particularity in the appended claims. A better understanding of the features and advantages of the present inventive concept will be obtained by reference to the following detailed description that sets forth illustrative examples, in which the principles of the disclosure are utilized, and the accompanying drawings of which:

FIG. 1 is a diagrammatic view of a wearable device, according to at least one instance of the present disclosure;

FIG. 2A is a diagrammatic view of a wearable device, according to at least one instance of the present disclosure;

FIG. 2B is a diagrammatic sectional view of a wearable device, according to at least one instance of the present disclosure;

FIG. 2C is a diagrammatic view of a spatially-resolved near-infrared spectroscopy (NIRS) sensor of a wearable device, according to at least one instance of the present disclosure;

FIG. 3 is a block diagram of a wearable device, according to at least one instance of the present disclosure;

FIG. 4 is a diagrammatic view of a wearable device system, according to at least one instance of the present disclosure;

FIG. 5. is a block diagram of a sleep management system, according to at least one instance of the present disclosure;

FIG. 6 is a flowchart of sleep management system operable with the wearable device system, according to at least one instance of the present disclosure;

FIG. 7 is a diagrammatic representation of a physiological response of hydration level to sleep, according to at least one instance of the present disclosure;

FIG. 8 is a flowchart of a breathing intervention exercise, according to at least one instance of the present disclosure.

### DETAILED DESCRIPTION

Examples and various features and advantageous details thereof are explained more fully with reference to the exemplary, and therefore non-limiting, examples illustrated in the accompanying drawings and detailed in the following description. Descriptions of known starting materials and processes can be omitted so as not to unnecessarily obscure the disclosure in detail. It should be understood, however, that the detailed description and the specific examples, while indicating the preferred examples, are given by way of illustration only and not by way of limitation. Various substitutions, modifications, additions and/or rearrangements within the spirit and/or scope of the underlying inventive concept will become apparent to those skilled in the art from this disclosure.

### I. Terminology

As used herein, the terms “comprises,” “comprising,” “includes,” “including,” “has,” “having” or any other variation thereof, are intended to cover a non-exclusive inclusion. For example, a process, product, article, or apparatus that comprises a list of elements is not necessarily limited only those elements but can include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term substantially, as used herein, is defined to be essentially conforming to the particular dimension, shape or other word that substantially modifies, such that the component need not be exact. For example, substantially cylindrical means that the object resembles a cylinder, but can have one or more deviations from a true cylinder.

The term “physiological” as used with respect to physiological sensors, physiological parameters, physiological changes, and the like herein refers to an aspect/characteristic of, or appropriate to, the healthy or normal functioning of a user, specifically with respect to the user's physical or emotional health or wellbeing.

Additionally, any examples or illustrations given herein are not to be regarded in any way as restrictions on, limits to, or express definitions of, any term or terms with which they are utilized. Instead these examples or illustrations are to be regarded as being described with respect to one particular example and as illustrative only. Those of ordinary skill in the art will appreciate that any term or terms with which these examples or illustrations are utilized encompass other examples as well as implementations and adaptations thereof which can or cannot be given therewith or elsewhere in the specification and all such examples are intended to be included within the scope of that term or terms. Language designating such non-limiting examples and illustrations includes, but is not limited to: “for example,” “for instance,” “e.g.,” “In some examples,” and the like.

Although the terms first, second, etc. can be used herein to describe various elements, components, regions, layers and/or sections, these elements, components, regions, layers and/or sections should not be limited by these terms. These terms are only used to distinguish one element, component, region, layer or section from another. Thus, a first element, component, region, layer or section discussed below could be termed a second element, component, region, layer or section without departing from the teachings of the present inventive concept.

### II. General Architecture

Wearable devices are configured to measure a data point and provide it a user in real-time without providing the user ways to improve the particular data and/or interpret the provided data. The disclosed wearable device offers physiological interventions at predetermined periods of time. The disclosed wearable device monitors whether the user is attempting an intervention and/or if the user is doing the intervention properly.

The systems and methods disclosed herein relate to monitoring and improving sleep through the use of a wearable device having one or more physiological sensors communicatively coupled therewith.

The wearable device can further be communicatively coupled with one or more context sensors operable to provide data relative to the one or more physiological sensors. The wearable device can detect one or more physiological indicators of a ready for sleep and suggest a sleep preparation exercise to the user if one or more of the physiological indicators exceed a predetermined threshold. The one or more indicators of sleep can be a user's physiologic state including a measurement of stress and/or arousal. In at least one instance, arousal can refer to the intensity of an emotion, emotional state, and/or emotional behavior. The wearable device can monitor compliance with the sleep preparation exercise and track whether the sleep preparation exercise successfully prepared the user for sleep via monitoring the one or more physiological indicators.

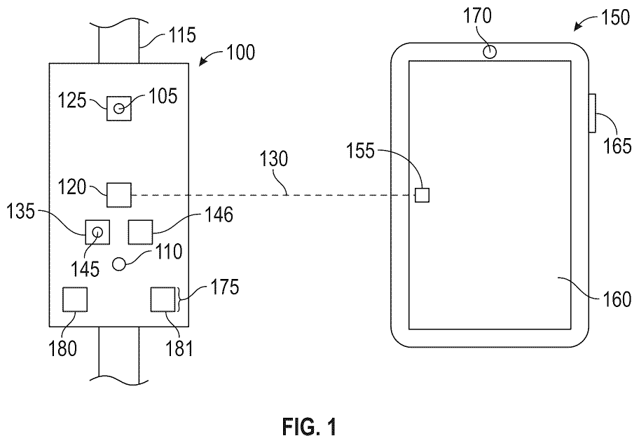

FIG. 1 illustrates a wearable device, according to an instance of the present disclosure. The wearable device 100 can be operably engaged with at least a portion of a user's body. In at least one instance the wearable device 100 can be operably engaged with the user via a band 115. In other instances, the wearable device 100 can be operably engaged with the user via a wearable clothing item (e.g. shirt, pants, shorts, compression sleeve, sock, ring, watch, hat, helmet, patch, etc.)

The portion of the user that the wearable device 100 is operable engaged with can be a plurality of locations including a muscle mass and/or tissue mass, including but not limited to a leg and/or arm of the user. In other instances, the portion of the user that the wearable device 100 is operably engaged with can include, but is not limited to, a wrist, a finger, a head, an ankle, neck, chest, and/or other portion of the user. In at least one instance, the portion of the user that the device is attached can be the wrist for accessibility and ease of use. In another instance, the portion of the user that the device is attached can be the finger for continuous wear. The wearable device 100 can be used with an optional output device 150, such as a smartphone (as shown), a smartwatch, computer, mobile phone, tablet, personal computing device, a generic electronic processing and displaying unit, cloud storage, and/or a remote data repository via a cellular network and/or wireless Internet connection (e.g. Wi-Fi).

The output device 150 can include a display 160 operable to provide a user information and/or data from the one or more physiological sensors (e.g. sensor 125, 135, 175). While the sensors are described herein as being one or more physiological sensors, it should be generally understood that the sensors of the wearable device disclosed herein can monitor any aspect of a user. The sensors, including the one or more physiological sensors, as described herein can include, but are not limited to, an electrodermal (EDA) sensor, a biomechanical sensor, a galvanic skin response (GSR) sensor, a photoplethysmography (PPG) sensor, an electrocardiogram (EKG), an inertial measurement sensor, an accelerometer, a gyroscope, a magnetometer, a global positioning system (GPS), a blood pressure (BP) sensor, a pulse oximetry (SpO2) sensor, a respiratory rate (RR) monitor, a temperature sensor, a humidity sensor, an audio sensor, an air quality sensor, a microphone, an environmental sensor (including but not limited to ambient noise, light, temperature, air quality, humidity, location, ultraviolet (UV) light exposure level, etc.), and/or any other sensor capable of measuring an aspect of a user and/or their environmental surroundings which may affect the user's physical and/or emotional health or wellbeing.

The output device 150 can include an input control device 165 operable to allow a user to change the display 160 and/or the information and/or data displayed thereon. In at least one instance, the input control device 165 can be a button and/or other actuatable element operable to allow an input to be received by the output device 150. In other instances, the input control device 165 can be a touch sensitive input device.

The output device 150 and the wearable device 100 can be communicatively coupled 130 via a transmitter/receiver 120, 155 disposed on the wearable device 100 and the output device 150, respectively. The communicative coupling 130 can be a two-way communication pathway allowing the wearable device 100 to provide information and/or data to the output device 150 and/or the display 160 while similarly allowing the output device 150 to request information and/or data from the wearable device.

One or more context sensors 170 can be disposed on the output device 150 and be operable to provide data regarding a user's ambient environment (e.g. temperature, humidity, light intensity, location, air quality, noise level, ultraviolet (UV) light level, screen usage (e.g. television, tablet, etc.), and/or smartphone usage etc.). The one or more context sensors 170 can provide comparative data for the one or more physiological sensors allowing the wearable device 100 to better understand the data measurements from the one or more physiological sensors. While the present disclosure illustrates the one or more context sensors 170 disposed on the output device 150, it is within the scope of this disclosure for the one or more context sensors to be coupled with and/or disposed on the wearable device 100, smart home sensors (e.g. smart thermostat, smart light switch, smart home hub, etc.).

The wearable device 100 can include one or more physiological sensors. The one or more physiological sensors can include, but are not limited to, an electrodemal sensor (EDA), a galvanic skin response (GSR) sensor, a photoplethysmography (PPG), an electrocardiogram (EKG), an inertial measurement sensor, an accelerometer, a gyroscope, a blood pressure sensor, a pulse oximetry (SpO₂) sensor, a respiratory rate monitor, a temperature sensor, a humidity sensor, an audio sensor, and combinations thereof.

The wearable device 100 can include a sensor 125 that is operable to determine a level of a biological indicator within tissue or blood vessels using near-infrared spectroscopy (NIRS). The sensor 125 can include an optical emitter 105 and/or an optical detector 110. The sensor 125 can uses one or more low-power lasers, light emitting diodes (LEDs) and/or quasi-monochromatic light sources and low-noise photodetecting electronics to determine an optical absorption. In another example, the sensor 125 can use a broad-spectrum optical source and a detector sensitive to the spectral components of light, such as a spectrometer, or a charge-coupled device (CCD) or other linear photodetector coupled with near-infrared optical filters.

The wearable device 100 can be configured to include a second sensor 135 operable to measure a photoplethysmography (PPG) of the user. The second sensor 135 can include an optical emitter 145 and/or an optical detector 146. The wearable device 100 can also include a third sensor 175 operable to measure electrocardiogram EKG) and/or derived systolic time intervals (STI) of the user. The third sensor 175 can include a first electrode 180 and/or a second electrode 181. The sensors 125, 135, 175 can each be a physiological sensor of the wearable device, collectively and/or individually. The wearable device 100 can include one or more physiological sensors including, but not limited to, sensors 125, 135, and/or 175, respectively.

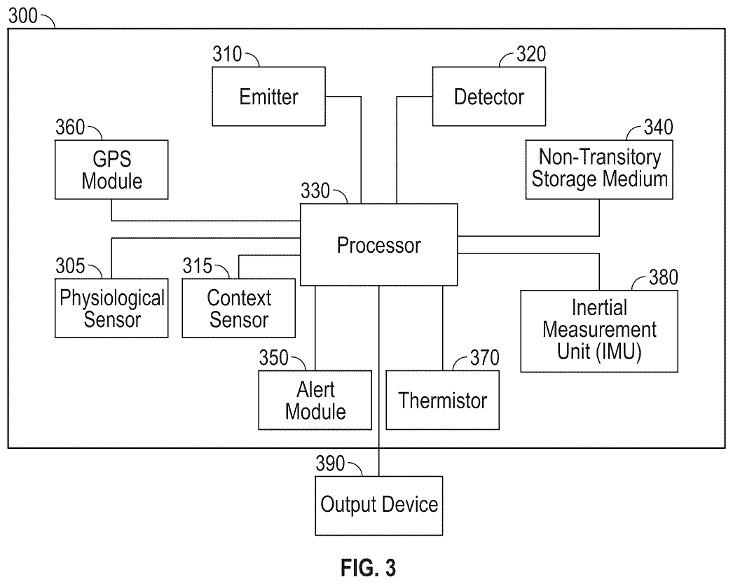

The sensors 125, 135, 175 in the device 100 can measure NIRS parameters, electrocardiogramography, and/or derived systolic time intervals (STI) of the user. The wearable device 100 also includes a processor (shown in FIG. 3) operable to analyze data generated by one or more of the sensors 125, 135, 175 to determine a physiological response and/or physiological change of a user.

In at least one instance, the processor is operable to determine biological indicators, including, but not limited to a relative percentage, a saturation level, an absolute concentration, a rate of change, an index relative to a training threshold, and a threshold. In other instance, the processor is operable to determine perfusion characteristics such as pulsatile rhythm, blood volume, vascular tone, muscle tone, and/or angiogenesis from total hemoglobin and/or water measurements.

The wearable device 100 can include a power supply, such as a battery, to supply power to one or more of the sensors 125, 135, 175 and/or other components in the wearable device 100. In at least one instance, the sensor 125 can be have a skin contact area of approximately 3.5 inches×2 inches. In other instances, the wearable device 100 can be sized to be on the user's wrist so that there is a skin contact area of approximately 1 inch×1 inch. In other instances, the wearable device 100 can be sized to be on the user's finger so that there is a skin contact area of approximately one quarter (¼) inch x one half (½) inch. Additionally, other dimensional skin areas are considered within the scope of this disclosure depending on the number of type of sensors operably implemented with the wearable device 100.

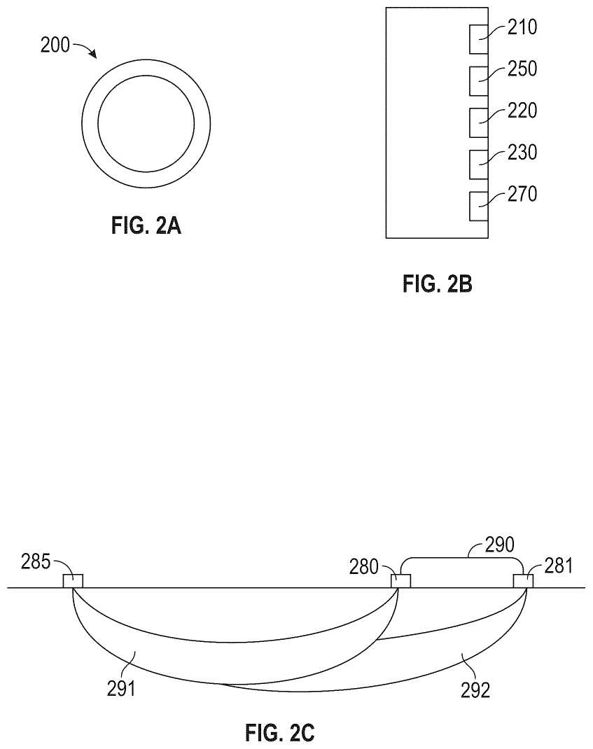

FIGS. 2A and 2B illustrates a wearable device having one or more optical physiological sensors, according to at least one instance of the present disclosure. The wearable device 200 can be configured to be worn on a finger of a user. In at least one example, the wearable device 200 can be optimized to a given finger for increased accuracy. The optimization can include physiological sensor selection, arrangement, orientation, and/or shape of the wearable device 200 to ensure proper fitment. In other instances, the wearable device 200 can be optimized based on the size, gender, and/or age of the user. In still other instances, a variety of the above optimizations can be implemented for a given device.

FIG. 2A illustrates a wearable device 200. FIG. 2B illustrates a cross-sectional of the wearable device 200, including emitters 220, 230, 250 and photodetector 210. The wearable device 200 also includes data and/or charging contacts 270. In at least one instance, the data and charging contacts 270 can be operable to electrically detect if the sensor is making contact with the skin of a user. The presence of multiple emitters 220, 230, and/or 250 on the wearable device 200 allows for spatially-resolved data gathering in real-time. The wearable device 200 can be configured to determine the optical absorption of chromophores, such as water, hemoglobin in its multiple forms, including oxyhemoglobin (HbO2), deoxyhemoglobin (HHb), oxymyoglobin, deoxymyoglobin, cytochrome c, lipids, melanins, lactate, glucose, or metabolites.

FIG. 2C illustrates a spatially-resolved NIRS sensor that can be included on the non-invasive wearable device 200, according to at least one instance of the disclosure. As shown in FIG. 2C, the spatially-resolved NIRS sensor can include light emitters 280 and 281 which emit light that is scattered and partially absorbed by the tissue. Each emitter 280, 281 can be configured to emit a single wavelength of light or a single range of wavelengths. In at least one example, each emitter 280, 281 can be configured to emit at least three wavelengths of light and/or at least three ranges of wavelengths. Each emitter 280, 281 can include one or more light emitting diodes (LEDs). Each emitter 280, 281 can include a low-powered laser, LED, or a quasi-monochromatic light source, and/or any combination thereof. Each emitter 280, 281 can also include a light filter.

A fraction of the light emitted by emitters 280 and 281 can be detected by photodetector 285, as illustrated by the parabolic or “banana shaped” light arcs 291 and 292. Emitters 280, 281, are separated by a known (e.g. predetermined) distance 290 and produce a signal that is later detected at photodetector 285. The detected signal is used to estimate the effective attenuation and absorption coefficients of the underlying tissue. In at least one instance, the known distance 290 is 12 mm. In other instances, the known distance can be selected based on a variety of factors, which can include the wavelength of the light, the tissue involved, and/or the age of the user.

The wearable device 200 disclosed herein can have different numbers of emitters and photodetectors without departing from the principles of the present disclosure. Further, the emitters and photodetectors can be interchanged without departing from the principles of the present disclosure. Additionally, the wavelengths produced by the LEDs can be the same for each emitter or can be different.

In at least one instance, the wearable device 200 can be used for the monitoring of one or more physiological parameters of a user. Use of the wearable device 200 is particularly relevant in endurance type sports, such as running, cycling, multisport competition, rowing, but can also be used in other physical activities. The device 200 can be configured to wirelessly measure real-time physiological parameters continuously throughout the day and/or night. The device 200 can be secured to a selected muscle group, such as the leg muscles of the vastus lateralis or gastrocnemius, or any area of the user where certain physiological parameters are best measured.

FIG. 3 illustrates the components of a wearable device 300 according to at least one instance of the present disclosure. As shown in FIG. 3, the wearable device 300 can include an emitter 310 and detector 320, which can be communicatively coupled to a processor 330. The processor 330 can be communicatively coupled to a non-transitory storage medium 340. The device 300 can be coupled to an output device 390.

The emitter 310 delivers light to the tissue and the detector 320 collects the optically attenuated signal that is back-scattered from the tissue. In at least one instance, the emitter 310 can be configured to emit at least three separate wavelengths of light. In another instance, the emitter 310 can be configured to emit at least three separate bands and/or ranges of wavelengths. In at least one instance, the emitter 310 can include one or more light emitting diodes (LEDs). The emitter 310 can also include a light filter. The emitter 310 can include a low-powered laser, LED, or a quasi-monochromatic light source, or any combination thereof. The emitter can emit light ranging from infrared to ultraviolet light. As indicated above, the present disclosure uses NIRS as a primary example and the other types of light can be implemented in other instances and the description as it relates to NIRS does not limit the present disclosure in any way to prevent the use of the other wavelengths of light.

The data generated by the detector 320 can be processed by the processor 330, such as a computer processor, according to instructions stored in the non-transitory storage medium 340 coupled to the processor. The processed data can be communicated to the output device 390 for storage or display to a user. The displayed processed data can be manipulated by the user using control buttons or touch screen controls on the output device 390.

The optical-electronic device 300 can include an alert module 350 operable to generate an alert including, but not limited to a suggested response to a physiological change. The processor 330 can send the alert to the output device 390 and/or the alert module 350 can send the alert directly to the output device 390. In at least one instance, the processor 330 can be operably arranged to send an alert to the output device 390 without the wearable device 300 including an alert module 350.

The alert can provide notice to a user, via a speaker or display on the output device 390, of a change in one or more physiological conditions or other parameter being monitored by the wearable device 300, or the alert can be used to provide an updated sleep preparation level to a user. In at least one instance, the alert can be manifested as an auditory signal, a visual signal, a vibratory signal, or combinations thereof. In at least one instance, an alert can be sent by the processor 330 when a predetermined physiological change occurs.

In at least one instance, the wearable device 300 can include a Global Positioning System (GPS) module 360 configured to determine geographic position and tagging the biological and/or physiological data with location-specific information. The wearable device 300 can also include a thermistor 370 and an IMU 380. The IMU 380 can be used to measure, for example, a gait performance of a walker and/or runner and/or a pedal kinematics of a cyclist, as well as one or more physiological parameters of a user. The thermistor 370 and IMU 380 can also serve as independent sensors configured to independently measure parameters of physiological threshold. The thermistor 370 and IMU 380 can also be used in further algorithms to process or filter the optical signal.

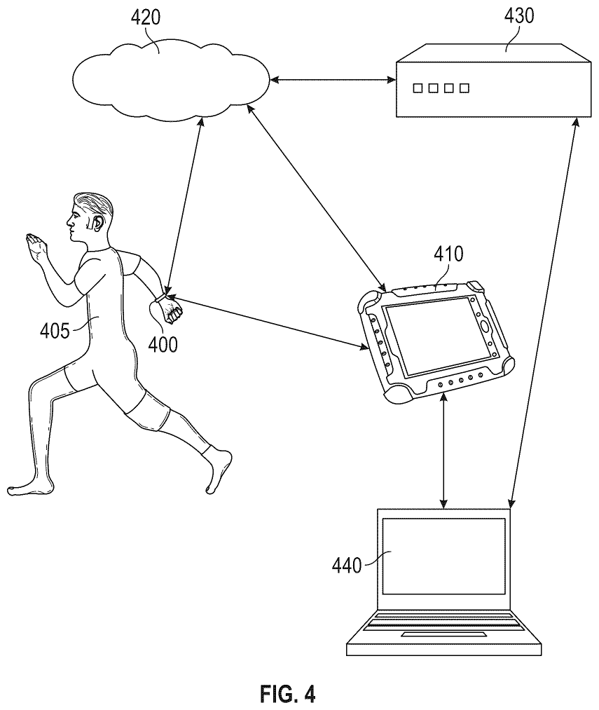

FIG. 4 illustrates an environment within which the wearable device can be implemented, according to at least one instance of the present disclosure. As shown in FIG. 4, the wearable device 400 is worn by a user to determine one or more biological and/or physiological indicator levels. The wearable device 400 is depicted as being worn on the wrist of a user 405; however, the wearable device 400 can be worn on any portion of the user suitable for monitoring biological and/or physiological indicator levels. The wearable device 400 can be used with an output device 410, such as a smartphone (as shown), a smart watch, computer, mobile phone, tablet, a generic electronic processing and/or displaying unit, cloud storage, and/or a remote data repository via a cellular network or wireless Internet connection.

As shown in FIG. 4, the wearable device 400 can communicatively couple with a output device 410 so that data collected by the wearable device 400 can be displayed and/or transferred to the output device 410 for communication of real-time biological and/or physiological data to the user 405. In at least one instance, an alert can be communicated from the device 400 to the output device 410 so that the user 405 can be notified of a biological and/or physiological event. Communication between the wearable device 400 and the output device 410 can be via a wireless technology, such as BLUETOOTH, infrared technology, or radio technology, and/or can be through a wire. Transfer of data between the wearable device 400 and/or the output device 410 can also be via removable storage media, such as a secure digital (SD) card. In at least one instance, a generic display unit can be substituted for the output device 410.

The wearable device 400 can communicatively couple with a personal computing device 440 and/or other device configured to store or display user-specific biological and/or physiological indicator data. The personal computing device 440 can include a desktop computer, laptop computer, tablet, smartphone, smart watch, or other similar device. Communication between the wearable device 400 and the personal computing device 440 can be via a wireless technology, such as BLUETOOTH®, infrared technology, or radio technology. In other instances, the communication between the wearable device 400 and the personal computing device 440 can be through a wire and/or other physical connection. Transfer of data between the optical-electronic device 400 and the personal computing device 440 can also be via removable storage media, such as an SD card.

The output device 410 can communicate with a server 430 via a network 420, allowing transfer of user-specific biological and/or physiological data to the server 430. The output device 410 can also communicate user-specific biological and/or physiological data and/or physiological data to cloud-based computer services or cloud-based data clusters via the network 420. The output device 410 can also synchronize user-specific biological and/or physiological data with a personal computing device 440 or other device configured to store or display user-specific biological and/or physiological data. The output device 410 can also synchronize user-specific biological and/or physiological data with a personal computing device 440 or other device configured to both store and display user-specific biological and/or physiological data. Alternatively, the personal computing device 440 can receive data from a server 430 and/or cloud-based computing service via the network 420.

The personal computing device 440 can communicate with a server 430 via a network 420, allowing the transfer of user-specific biological and/or physiological data to the server 430. The personal computing device 440 can also communicate user-specific biological and/or physiological data to cloud-based computer services and/or cloud-based data clusters via the network 420. The personal computing device 440 can also synchronize user-specific biological and/or physiological data with the output device 410 and/or other device configured to store or display user-specific biological and/or physiological data.

The wearable device 400 can also directly communicate data via the network 420 to a server 430 or cloud-based computing and data storage service. In at least one instance, the wearable device 400 can include a GPS module configured to communicate with GPS satellites (not shown) to obtain geographic position information.

The wearable device 400 can be used by itself and/or in combination with other electronic devices and/or context sensors. The context sensors can include, but are not limited to, sensors coupled with electronic devices other than the wearable device 400 including smart devices used both inside and outside of a home. In at least one instance, the wearable device 400 can be used in combination with heart rate (HR) biosensor devices, foot pod biosensor devices, and/or power meter biosensor devices. In at least one instance, the wearable device 400 can also be used in combination with ANT+™ wireless technology and devices that use ANT+™ wireless technology. The wearable device 400 can be used to aggregate data collected by other biosensors including data collected by devices that use ANT+™ technologies. Aggregation of the biosensor data can be via a wireless technology, such as BLUETOOTH®, infrared technology, or radio technology, or can be through a wire.

The biosensor data aggregated by the wearable device 400 can be communicated via a network 420 to a server 430 or to cloud-based computer services or cloud-based data clusters. The aggregated biosensor data can also be communicated from the wearable device 400 to the output device 410 or personal computing device 440.

In at least one instance, the wearable device 400 can employ machine learning algorithms by comparing data collected in real-time with data for the same user previously stored on a server 430, output device 410, and/or in a cloud-based storage service. In other instances, the wearable device 400 can compare data collected in real-time with data for other users stored on the server 430 and/or in cloud based storage service. The machine learning algorithm can also be performed on or by any one of the output device 410, cloud-based computer service, server 430, and/or personal computing device 440, and/or any combination thereof.

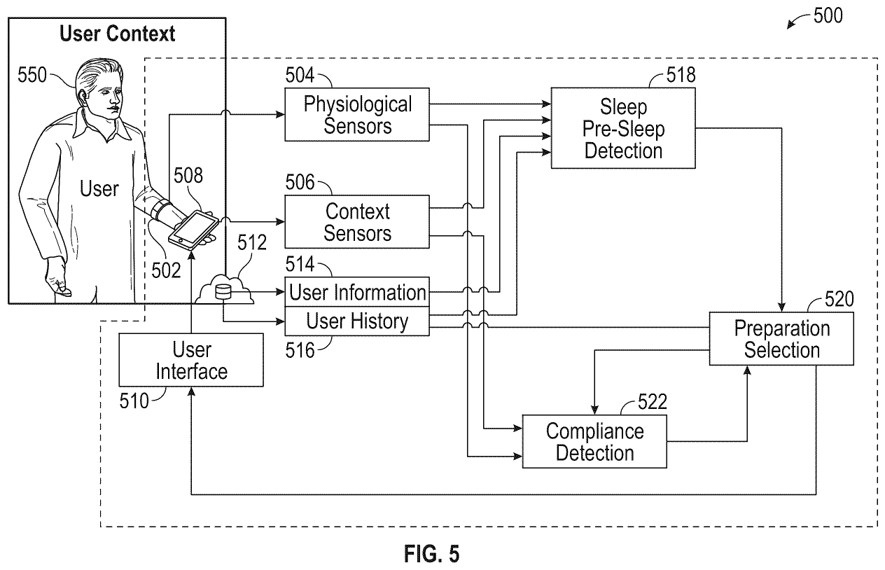

FIG. 5 illustrates an example wearable device system operable to detect and manage a sleep preparation level of a user. The wearable device 502 can include one or more physiological sensors 504 operably engaged with the user and operably coupled with the wearable device system 500. The one or more physiological sensors 504 can include an electrodemal sensor (EDA) sensor, a photoplethysmography (PPG) sensor, an electrocardiogram (EKG) sensor, an inertial measurement sensor, an accelerometer, a gyroscope, a blood pressure sensor, a pulse oximetry (SpO2) sensor, a respiratory rate monitor, a thermometer, a humidity sensor, an audio sensor, and/or combinations thereof. The one or more physiological sensor 504 can be an optical sensor including active and/or passive camera systems operable to quantify blood pulse volume, blood pressure, heart rate, heart rate variability, and/or optically opaque compounds (e.g. hemoglobin, etc.).

Thermal systems can be operable to measure temperature via infrared systems and/or thermocouples. Sweat quantification systems can be galvanic skin response (GSR) and/or EDA. Pressure system can be implemented to monitor blood pressure, and motion system can be implemented to monitor user 550 movement including, but not limited to, IMU, accelerometer, gyroscope, magnetometer, and/or GPS.

The wearable device 502 can be a watch, wristband, ring, necklace, clothing (e.g. shirt, sock, underwear, bra, compression sleeve, etc.), adhesive patch, continuous glucose monitors (CGM), other medical equipment, and/or combinations thereof. Additionally, the wearable device 502 can be implemented to include one or more of the features described above with respect to wearable devices illustrated in FIGS. 1-4.

The wearable device system 500 can be communicatively coupled with one or more context sensors 506 operably coupled with the wearable device 502. The one or more context sensors 506 can provide the wearable device system 500 with information about a user's ambient environment and/or location. The one or more context sensors 506 can provide ambient temperature, ambient light intensity, ambient humidity, and/or location. The one or more context sensors 506 can be disposed on the wearable device 502 and/or communicatively coupled with the wearable device 502. In at least one instance, the one or more context sensors 506 can include a smartphone operable to provide location information of the user. In other instances, the one or more context sensors 506 can include a smart thermostat operable provide ambient temperature information (e.g. room temperature), a smart light switch operable to provide ambient light intensity information, a smart hub operable to provide location information within a home, bathroom fixtures (e.g. scale, mirror, toilet with sensors, etc.), smart microphones, smart refrigerators, vehicles, and/or combinations thereof.

The wearable device system 500 can utilized the one or more context sensors 502 to appropriate characterize and/or provide prospective to the physiological data of the one or more physiological sensors 504.

The wearable device system 500 can further include a display 508 operable to engage with the user 550. In at least one instance, the display 508 can be a user's smartphone and can be independent of but communicatively coupled with the wearable device 502. The display 508 can provide a user interface 510 through which a user 550 interacts with the wearable device system 500.

A server 512 can be communicatively coupled with the wearable device 502 and can be operable to store user information 514 and/or user history 516. The user information 514 and/or user history 516 can be include input personal information about the user (e.g. height, weight, age, gender, medical history, etc.) and/or stored measurements obtained from the one or more physiological sensors 504 and/or the one or more context sensors 506. The server 512 can be a conventional physical server and/or a cloud-based server storage solution.

The wearable device 502 can determine a sleep and/or pre-sleep detection 518 via measurements from the one or more physiological sensors 504 and/or the one or more context sensors 506. The sleep or pre-sleep detection 518 can be indicated by changes in one or more physiological responses by the user 550 while accounting for the user's environment through the one or more context sensors 506. In at least one instance, the sleep or pre-sleep detection 518 can be determined by a location within a user's home via one or more context sensors 506 (e.g. proximity to a nightstand and/or docking station). In other instances, the sleep or pre-sleep detection 518 can be determined via ambient light, user's body orientation (e.g. laying down), heart rate, respiration rate, low movement, circadian body temperature fluctuations, time, user interaction, and/or combinations thereof.

The sleep or pre-sleep detection 518 can have a predetermined threshold for a sleep index in view of the user information 514 and/or user history 516 and/or collective user data obtained through a cloud storage solution.

Upon detection of a sleep and/or pre-sleep above the predetermined threshold, the wearable device 502 can offer a sleep preparation selection 520. In at least one instance, the sleep preparation selection 520 can a few options operable to prepare the user for sleep and allow the user to select a desired sleep preparation 520. In other instances, the sleep preparation selection 520 can be a single option operable to improve a user's sleep index. The sleep and/or pre-sleep threshold can include a stress threshold operable to determine if a user is too stressed and/or not relaxed enough to obtain optimum sleep. A user may be stressed and/or not relaxed enough due to one or more physiological elements and/or one or more environmental elements including, but not limited to, breathing rate, heart rate, movement, biomechanics, body position, limb position, phone usage, screen time, and/or combinations thereof. Upon detection of a stress or pre-stress above the predetermined threshold, the wearable device 502 can offer a sleep preparation selection 520 operable to reduce stress. In at least one instance, the sleep preparation selection 520 can a few options operable to reduce a stress index of the user as measured by the one or more physiological sensors and allow the user to select a desired stress intervention 520. In other instances, the sleep preparation selection 520 can be a single option operable to reduce a stress index

Stress can be measured and/or determined from the one or more physiological sensors by determining a physiological change and/or combination of physiological changes experienced by a user. Examples of indications of stress include, but are not limited to, increased heart rate (not caused by physical activity), increases in breathing rate, decrease in skin temperature due to sweating and/or peripheral vasoconstriction without a decrease in ambient temperature (via the one or more context sensors 506), increases in glucose without recent food ingestion, increases in skin conductivity response (SCR) and rate of sweat glad activation without physical activity, decrease in peripheral perfusion, decrease in heart rate variability (e.g. a more regular heart beat), increase in blood pressure, movement deviation away from a normal patter (e.g. pacing), changes in vocalizations (e.g. shouting, yelling, and/or tone), and/or combinations thereof.

As the user participates in the sleep preparation selection 520, the wearable device 502 can have compliance detection 522 to determine if the user is participating in the sleep preparation selection 520 appropriately. In at least one instance, the sleep preparation selection 520 can be a box breathing exercise and the compliance detection 522 can monitor the user's 550 breathing pattern and/or respiration rate to determine if the user is following the box breathing exercise. In other instances, the sleep preparation selection 520 can be meditating and/or listening to music and the one or more context sensors 506 and/or the one or more physiological sensors 504 can be monitored to determine if the ambient noise, light, and/or respiration rate, etc. changed, thereby indicating the user 550 is meditating and/or listening to music. The sleep preparation selection 520 can alternatively be meditation, walk, exercise, movement, music, videos, journaling exercise, biofeedback, psychotherapy (including cognitive behavioral therapy (CBT)), acts of kindness, social connections and/or interactions

If the compliance detection 522 determine the user 550 is not complying with the sleep preparation selection 520, the preparation selection 520 can be continued and/or repeated until the compliance detection 522 determines the user 550 has succeeded in completing the sleep preparation selection 520.

The wearable device 502 monitors the sleep or pre-sleep detection 518 before, during, and/or after the sleep preparation selection 520, and can determines if the sleep or pre-sleep detection dropped below the predetermined threshold following the sleep preparation selection 520. If the sleep readiness did not drop below the predetermined threshold, the user can be recommended to complete another sleep preparation selection 520 exercise. In at least one instance, the subsequent sleep preparation selection 520 can be a new exercise or activity. Similarly, if the stress index did not drop below the predetermined threshold, the user 550 can be recommended to complete another sleep preparation selection 520.

The wearable device system 500 can monitor, track, and learn which sleep preparation selection 520 work for a particular user 550 and recommend them more regularly than other stress intervention selections 520. In at least one instance, the wearable device system 500 can be operable to determine different types of stress indicated by the one or more physiological sensors 502, and recommend varying sleep preparation selection 520 based on the type of stress detected during sleep preparation. The types of stress can be determined based on the user physiological response as measured by the one or more physiological sensors 504 (e.g. heart rate, temperature, perspiration, etc.).

In some instances, the user interface 510 can be operable to guide the user 550 through the sleep preparation selection 520 by illustrating a video, diagram, and/or other graphic. The user interface 520 can provide the user 550 instructions and/or demonstration for a sleep preparation selection 520. In at least one instance, the user interface 510 can provide a box breathing video demonstrating how the technique is performed, while also indicating when a user 550 inhale and exhale, as appropriate. The user interface 510 can thus assist in ensuring compliance with the intervention selection.

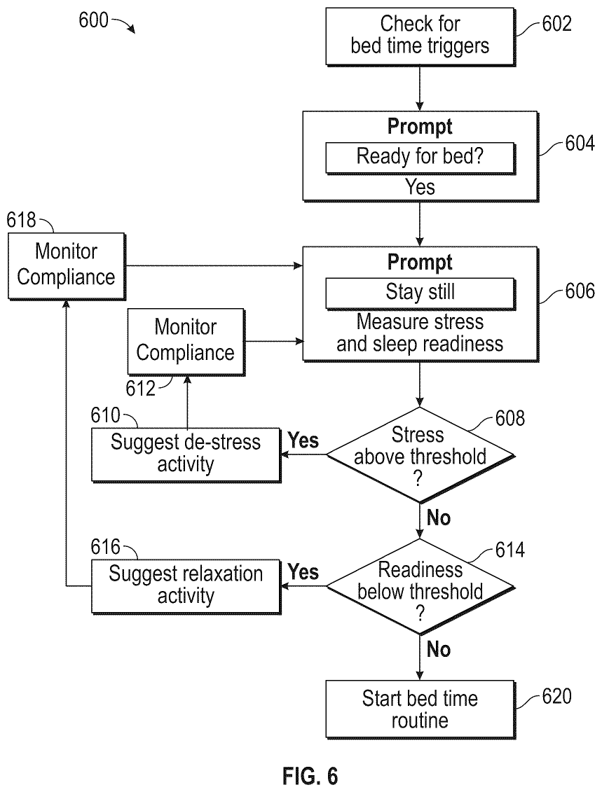

FIG. 6 illustrates a flowchart of a sleep preparation and/or management system operable to be implemented with a wearable device, according to at least one instance of the present disclosure. A wearable device having one or more physiological sensors operably coupled therewith can obtain one or more physiological measurements from the one or more physiological sensors, for example those described above with respect to FIGS. 1-5.

The sleep preparation and/or management system 600 can be operable to check for one or more bedtime triggers 602, via the one or more physiological sensors and/or the one or more context sensors. The one or more bedtime triggers 602 can include, but are not limited to, low ambient light levels, low ambient noise levels, low movement, circadian body temperature fluctuations, time of day (e.g. average of a predetermined number of prior days at which user went to sleep, and/or average of predetermined number of specific days, Monday, at which the user went to sleep), user interaction (e.g. user indicates going to bed), and/or proximity to bed (e.g. determined via BLUETOOTH® proximity to a charging stand).

In view of the one or more bedtime triggers 602, the sleep preparation and/or management system 600 can inquire whether a user is ready for bed 604. The ready for bed prompt 604 can be an auditory and/or visual alert presented to the user on the wearable device and/or a display of the wearable device system. If the user indicates they are not ready for bed, the sleep preparation and/or management system 600 can terminate because the user does not intend to go to sleep. In at least one instance, the wearable device and/or related system train and/or learn a user's sleep indication based on the user declining ready for bed. The wearable device and/or related system can determine the user's sleep pattern, sleep timing, etc. from prompting a user and being declined.

If the user indicates a ready for bed 604, wearable device can instruct a user to stay still measure a sleep readiness and/or a stress index 606 operable to determine whether the user is physiologically ready for sleep. The wearable device can determine if a stress index level 608 is above a predetermined threshold. If the stress index level exceeds a predetermined threshold, a stress intervention sleep preparation activity can be suggested 610 to the user. The predetermined threshold of the stress index can be relative to a user's sleep preparation, such that the user may not actively feel stressed, but rather is too stressed to sleep because their physiological measurements index a stress index exceeding the predetermined threshold. Thus, the predetermined threshold of the stress index can be lower than a physical and/or mental representation of stress, but sufficient to indicate a physiological stress via the one or more physiological sensors.

The stress intervention sleep preparation activity can be a breathing exercise (e.g. a box breathing exercise) operable to reduce the physiological stress index measured by the wearable device. The stress intervention sleep preparation activity can be operable to reduce one or more physiological measurement, thereby reducing the stress index below the predetermined threshold. The sleep preparation activities and/or exercises 618 can be a breathing exercise (e.g. box breathing), listening to music, meditating, reducing ambient light, reducing ambient noise, and/or the like. As can be appreciated in FIG. 6, the wearable device can monitor compliance 612 with instructions, recommendations, and/or process of the stress intervention sleep preparation activity. In at least one instance, the wearable device monitors compliance 12 with the box breathing exercise by monitoring a user's respiration rate and/or breathing pattern by the one or more physiological sensors.

If the stress index falls below the predetermined threshold, the sleep preparation and/or management system 600 can determine whether sleep readiness 614 is below a predetermined threshold. Sleep readiness can be determined based on one or more physiological sensors and/or one or more context sensors. In at least one instance, an elevated heart rate and high ambient light can indicate a user is not ready for sleep. In other instances, a relaxed heart rate and low ambient light can indicate a user is ready for sleep.

If the sleep readiness 614 exceeds a predetermined threshold, the sleep preparation and/or management system 600 can suggest a relaxation intervention 616 to improve the user's sleep readiness. The relaxation intervention can be a breathing exercise, a meditation exercise, a calming exercise, and/or any other relaxation intervention. In at least one instance, the stress intervention sleep preparation activity and/or the relaxation intervention can be the same activity. In other instances, the stress intervention sleep preparation activity and/or the relaxation intervention can be different activities.

The relaxation intervention can include a meditation, yoga, stretching, and/or other relaxing activity to calm the user and prepare them for sleep. In at least one instance, the relaxation intervention can be listening to calming sounds for a predetermined period of time. The sleep preparation and/or management system 600 can monitor compliance 618 with the relaxation intervention.

After the sleep preparation and/or management system 600 has achieved a stress index 608 below the predetermined threshold and a sleep readiness below the predetermined threshold, the user can be instructed to begin their bedtime routine. The sleep preparation and/or management system 600 can monitor the wearable device during the bedtime routine to insure the user does not exceed the stress index predetermined threshold and/or the sleep readiness predetermined threshold during this routine. In some instances, the sleep preparation and/or management system 600 can determine if a user's bedtime routine requires adjustment due to elevated stress index and/or poor sleep readiness.

The sleep preparation and/or management system 600 can train and/or learn, via machine learning algorithms and/or the one or more processors, which de-stressing activities and/or relaxation interventions are successful in de-stressing and/or relaxing the user to improve the suggestion of stress intervention sleep preparation activity 610 and/or relaxation interventions 616.

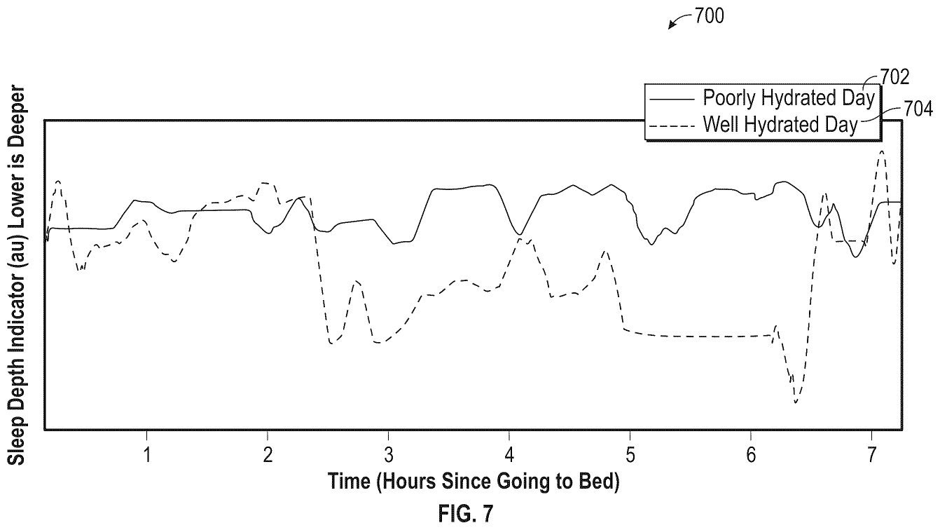

FIG. 7 illustrates a hydration relationship with sleep, according to at least one instance of the present disclosure. The hydration level 700 can be tracked in relation to a user's sleep depth. A sleep depth indicator can quantify the quality of a user's sleep using reference to one or more physiological sensors (e.g. motion, brain waves, oxygen level in blood, heart rate, breathing rate, eye movement, and/or leg movement).

As can be appreciated in FIG. 7, a poorly hydrated day 702 can show relatively low sleep depth indicator throughout the duration of user sleep, while a well hydrated day 704 can show a sleep depth indicator that increases during the user sleep, thus indicating a deep sleep obtained by the user. Therefore, there appears to be a strong correlation between poor hydration and poor sleep, which can be utilized by the wearable device. In at least one instance, the sleep readiness activity can include drinking a glass of water to improve hydration and thus ultimately improve sleep.

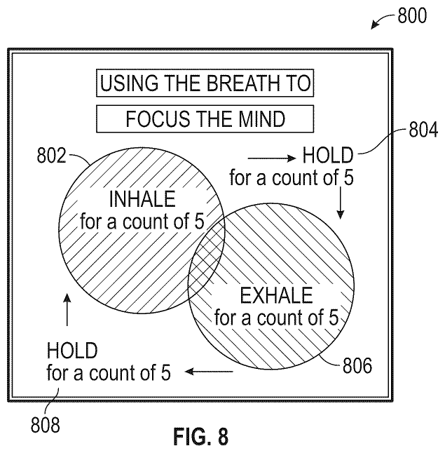

FIG. 8 illustrates a box breathing stress intervention activity, according to at least one instance of the present disclosure. A box breathing exercise 800 can be utilized to as a stress intervention activity following detection by the wearable device that a user is experiencing stress. The box breathing exercise 800 include an inhale 802 portion followed by an exhale portion 804. The inhale portion 802 can instruct the under to inhale for a predetermined number of seconds (e.g. to a count of 5) and then to a hold portion 804 in which the inhaled breath can be maintained for a similar predetermined number of seconds. The user can then be instructed to proceed to the exhale portion 806 in which the user exhales for a predetermined number of seconds (e.g. to a count of 5) and then to a hold portion 808 in which the inhaled breath can be maintained for a similar predetermined number of seconds.

While box breathing is illustrated as a specific example of a breathing exercise, it is within the scope of this disclosure to implement any number of breathing exercises including, but not limited to, pursed lip breathing, belly breathing, breath focus, lion's breath, alternate nostril breathing, equal breathing, resonant breathing, sitali breath, deep breathing, and/or humming bee breath. The wearable device can be operable provide instruction on the breathing exercise and/or monitor the user's compliance with the breathing exercise through respiration monitoring via the one or more physiological sensors.

While preferred examples of the present inventive concept have been shown and described herein, it will be obvious to those skilled in the art that such examples are provided by way of example only. Numerous variations, changes, and substitutions will now occur to those skilled in the art without departing from the disclosure. It should be understood that various alternatives to the examples of the disclosure described herein can be employed in practicing the disclosure. It is intended that the following claims define the scope of the disclosure and that methods and structures within the scope of these claims and their equivalents be covered thereby.

### Statement Bank

Statement 1: A sleep management apparatus, comprising: a wearable device having one or more physiological sensors operably engaged with a body of a user; one or more processors communicatively coupled with the wearable device, the one or more processors having a memory storing instructions when executed operable to: detect one or more indicators of a bedtime; measure one or more physiological indicators predictive of a ready for sleep condition of the user; suggest, when one or more of the one or more physiological indicators exceed a predetermined threshold, a sleep preparation exercise; and track an effectiveness of the sleep preparation exercise.

Statement 2: The sleep management apparatus of Statement 1, wherein the one or more indicators of the bedtime are a location, a time, a user position, ambient light, and/or combinations thereof.

Statement 3: The sleep management apparatus of Statement 1 or Statement 2, wherein the one or more indicators of the bedtime are associated with one or more context sensors.

Statement 4: The sleep management apparatus of any one of Statements 1-3, wherein the one or more context sensors are a user's phone, a docking station, and/or a smart home device.

Statement 5: The sleep management apparatus of any one of Statements 1-4, wherein the effectiveness of the sleep preparation exercise is relative to the one or more physiological indicators of ready for sleep.

Statement 6: The sleep management apparatus of any one of Statements 1-5, wherein the effectiveness of the sleep preparation exercise is a sleep quality index tracked during sleep by the one or more physiological sensors.

Statement 7: The sleep management apparatus of any one of Statements 1-6, wherein the sleep preparation exercise is adjusted based on the effectiveness of the sleep preparation exercise.

Statement 8: The sleep management apparatus of any one of Statements 1-7, wherein the one or more physiological sensors are operable to detect one or more of skin temperature, heart rate, heart rate variability, blood pulse volume, blood pressure, and/or perspiration.

Statement 9: The sleep management apparatus of any one of Statements 1-8, wherein the one or more physiological indicators of sleep are one or more of skin temperature, heart rate, heart rate variability, blood pulse volume, blood pressure, perspiration, breathing rate, an electrodemal (EDA), a galvanic skin response (GSR), blood oxygen (SpO₂) movement, biomechanics, body position, limb position, phone usage, and/or screen usage.

Statement 10: The sleep management apparatus of any one of Statements 1-9, wherein the sleep preparation exercise is a breathing exercise having a predetermined sequence of inhale and/or exhale patterns.

Statement 11: The sleep management apparatus of any one of Statements 1-10, wherein the sleep preparation exercise is a meditation exercise, breathing exercise, sleep story, journaling exercise, and/or biofeedback.

Statement 12: The sleep management apparatus of any one of Statements 1-11, wherein the sleep preparation exercise modifies one or more environmental factors.

Statement 13: The sleep management apparatus of any one of Statements 1-12, wherein the one or more environmental factors is temperature, noise, and/or ambient light.

Statement 14: A sleep management method, the method comprising: detecting, via one or more physiological sensors, one or more indicators of a bedtime; measuring, via the one or more physiological sensors, one or more physiological indicators predictive of a ready for sleep condition of the user; suggesting, if one or more of the one or more physiological indicators exceed a predetermined threshold, a sleep preparation exercise; and tracking an effectiveness of the sleep preparation exercise.

Statement 15: The method of Statement 14, wherein the one or more indicators of the bedtime are a location, a time, a user position, ambient light, and/or combinations thereof.

Statement 16: The method of Statement 14 or Statement 15, wherein the one or more indicators of the bedtime are associated with one or more context sensors, wherein the one or more context sensors are a user's phone, a docking station, and/or a smart home device.

Statement 17: The method of any one of Statements 14-16, wherein the effectiveness of the sleep preparation exercise is relative to the one or more physiological indicators of ready for sleep.

Statement 18: The method of any one of Statements 14-17, wherein the effectiveness of the sleep preparation exercise is a sleep quality index tracked during sleep by the one or more physiological sensors.

Statement 19: The method of any one of Statements 14-18, wherein the sleep preparation exercise is adjusted based on the effectiveness of the sleep preparation exercise.

Statement 20: The method of any one of Statements 14-19, wherein the sleep preparation exercise modifies one or more environmental factors, wherein the one or more environmental factors is temperature, noise, and/or ambient light.

What is claimed is:

1. A sleep management ring, comprising:
a ring including one or more physiological sensors configured to detect one or more physiological indicators of a user;
an output device including one or more context sensors; and
one or more processors communicatively coupled with the ring and the output device, the one or more processors having a memory storing instructions when executed operable to:
receive one or more indicators of a bedtime from the one or more context sensors;
receive the one or more physiological indicators from the one or more physiological sensors;
recommend, when one or more of the one or more physiological indicators and/or the one or more indicators of a bedtime exceed a predetermined threshold, a sleep preparation exercise for a user to perform;
determine a change in the one or more physiological parameters and/or the one or more indicators of the bedtime after recording the sleep preparation exercise; and
adjust the sleep preparation exercise based on one of the effectiveness of the sleep preparation exercise and the one or more physiological indicators as measured by the one or more physiological sensors of the wearable device.
